(19) United States
(12) Patent Application Publication
(10) Pub. No.: US 2021/0338987 A1
(43) Pub. Date: Nov. 4, 2021

(54) FLUID VESSEL INSERTION DEVICE WITH AUTOMATIC NEEDLE RETRACTION

(71) Applicant: ACADEMISCH ZIEKENHUIS LEIDEN (H.O.D.N. LUMC), Leiden (NL)

(72) Inventors: Wouter Jan GEELHOED, Leiden (NL); Joris Ivo ROTMANS, Leiden (NL)

(21) Appl. No.: 17/281,339

(22) PCT Filed: Oct. 4, 2019
(86) PCT No.: PCT/NL2019/050665

§ 371 Date: Mar. 30, 2021

(30) Foreign Application Priority Data
Oct. 5, 2018 (NL) 2021769

(51) Int. Cl. A61M 25/06; A61M 5/158

(52) U.S. Cl. CPC A61M 25/0631; A61M 25/0606; A61M 5/158; A61M 2005/1583; A61M 2205/0238; A61M 2210/12; A61M 2205/0227

(57) ABSTRACT

The present invention relates to a fluid vessel insertion device comprising a) a puncture member arranged for location within a cannulation sheet and comprising an interior lumen; b) a retractor arranged to actuate the puncture member relative to the cannulation sheet in use between i) a first situation in which a distal end of the puncture member protrudes beyond an end of the cannulation sheet, and ii) a second situation in which the distal end of the puncture member is within the cannulation sheet; c) a chamber in fluid communication with an exterior of the device only via the lumen and distal end of the puncture member; and d) a sheet of material located in the chamber to prevent the proximal end of the puncture member from moving; wherein the distal end of the puncture member is configured as a puncturing end having a distal n end opening for insertion into a fluid vessel when in use, and wherein the retractor triggers retraction of the puncture member between the first and the second situation automatically in response to wetting of the sheet of material by a fluid entering the puncture member lumen, whereby the sheet of material is comprising a material whose tensile strength is reduced upon contact with the fluid, and breaks under the pressure of the retractor.

The present invention relates to a fluid insertion device with automatic needle retraction, more particular to a device for insertion into a vessel to allow fluid communication with the interior of the vessel. Such devices may be used in medical applications although it is not limited thereto. The present invention furthermore relates to process for safely placing a cannulation sheet in a vessel.

Within the medical field, there are several situations in which it is necessary to puncture fluid vessels for delivery of fluid to, or removal of fluid from, a fluid vessel in the body. Intravenous cannulation is a common example of such a medical procedure. Intravenous cannulation is expected routinely to fail in 20-30% of initial attempts and can prove to be problematic for certain groups of patients or inexperienced operators. The problems are particularly aggravated because most patients requiring extracorporeal haemodialysis must undergo treatment as frequently as three to four times a week. This means that if every venepuncture were completely successful, a patient would need to undergo already from 6 to 8 venipunctures or cannulations each week.

It is well known that the duration and well-functioning of a fistula created by venepuncture is inversely related to the number of venipunctures. Tissue repeatedly subjected to the trauma of venipuncture is much more susceptible to infection, clotting hematoma, and thrombophlebitis. It is commonly found in patients who have experienced a number of venipunctures, that the tissue surrounding the most accessible veins develop large hematomas which obscure the veins, making successful venipunctures extremely difficult because of insufficient blood flow in the damaged blood vessels.

For an operator it is difficult to correctly interpret the point at which the vessel is punctured, even for the most experienced ones. That might be because the walls of fluid vessels in the body, such as blood veins, often do not offer a significant increase in resistance to the travel of the needle compared to the surrounding tissue. Furthermore, the depth of the vessel might be relatively small, so there is little room for error when attempting to position the needle end within the vessel. Even if the operator does correctly place the needle in the vessel, there remains the unintentional movement of the operator or a patient thereafter could cause further puncturing of the vessel, for example through the opposing vessel wall, resulting in a failed cannulation attempt.

There is a lot of effort going on in to provide devices which present feedback to an operator to aid in determining the location of the needle within the body. Such devices are known for instance from US 2018/0064465 A1, wherein a fluid vessel insertion device is described for use during positioning of a sleeve in a desired fluid vessel, such as, for example, during intravenous cannulation. Here the needle retracts in response to fluid pressure at the end of the needle.

US 2012/0150118 describes devices and methods for insertion of a catheter into a vessel. The actuator is designed to urge the needle to slide proximally relative to the dilator on contact of the actuator with a warm or aqueous fluid, e.g., blood flowing from the needle bore.

An objective of this invention is to provide a fluid insertion device which allows for improved accuracy and/or reliability of insertion.

It is a further object of this invention to provide a device that improves haemodialysis, arterial, and venous cannulation with respect to incorrect cannulation resulting in hematoma formation.

It is a further object of this invention to decrease the occurrence of major fistula infiltration, in which a fistula is punctured with a resulting hematoma, to a frequency below 5.2% per patient per year, see for example T. Lee et all, Am. J. Kidney. Dis, 2006, 47(6): p. 1020-6.

Another object of this invention is improving the comfort of the patents when undergoing dialysis.

A still further object is improving the accuracy in retraction of the needle. Pressure differences might be the result of more than entering a fluid vessel only.

It is a still further object of the present invention to provide in a solution that is economically attractive.

These and other features and characteristics of fluid vessel insertion devices according to the present invention will be apparent from the following description and accompanying illustration of typical embodiments thereof.

The present invention relates to a fluid vessel insertion device comprising

a) a puncture member arranged for location within a cannulation sheet and comprising an interior lumen;

b) a retractor arranged to actuate the puncture member relative to the cannulation sheet in use between

i) a first situation in which a distal end of the puncture member protrudes beyond an end of the cannulation sheet, and

ii) a second situation in which the distal end of the puncture member is within the cannulation sheet;

c) a chamber in fluid communication with an exterior of the device only via the lumen and distal end of the puncture member; and

d) a sheet of material located in the chamber to prevent the proximal end of the puncture member from moving;

wherein the distal end of the puncture member is configured as a puncturing end having a distal end opening for insertion into a fluid vessel when in use, and

wherein the retractor triggers retraction of the puncture member between the first and the second situation automatically in response to wetting of the sheet of material by a fluid entering the puncture member lumen, whereby the sheet of material is comprising a material whose tensile strength is reduced upon contact with the fluid, and breaks under the pressure of the retractor.

The determination of a fluid wetting a material as the trigger for retraction of the puncture member is advantageous since it allows accurate assessment of the point of entry of the puncture member into a vessel comprising a fluid, for example a blood vessel. The determination is advantageously not reliant on feedback pertaining to the resistance of the vessel wall to puncturing.

Furthermore, the retraction of the puncture member relative to the sleeve requires minimal movement and/or retraction force. Thus the sleeve can reliably remain in the desired position within a vessel with minimal disturbance.

The sheet of material, that is comprising a material whose tensile strength is reduced upon contact with a liquid can be any material that has an initial tensile strength that is sufficiently strong to withhold a piston head in a retractor, and of which the tensile strength reduces upon contact with a liquid in such a way that the piston head moves sufficiently to withdraw the puncture member inside of a cannulation sheet.

Preferably, the sheet of material has a wet strength of less than 10%, more preferably less than 8%. The wet strength of a material is often referred to as relative wet-strength, the ratio between wet strength and dry strength.

Preferably, the sheet of material is comprising a wettable membrane. More preferably, the sheet of material comprises nylon and/or cellulose based membranes. Other options are that the material comprises a sugar-based, starch-based membrane, or a hydrofilm, or a cellulose paper. Preferably the material may be subjected to sterilisation essentially without affecting the tensile strength. More preferably, the entire device may be sterilised, and this packaged prior to use. Alternatively, the membrane may be replaced separately, and the present invention furthermore also relates to a wettable membrane for use in the subject device, whereby the device is configured to be reused after sterilisation.

Alternative to the sheet of material that breaks upon contact with a fluid, it might be that the sheet of material has a sponge-like consistency or different shape, e.g. baton or elongate stick shape, that is initially sufficiently hard to withstand the piston pressure, but is dented or deformed upon contact with a fluid, resulting in that the piston head sufficiently moves to transport the puncture device into the cannulation sheet.

It is, however, preferred to use sheet-like materials. A sheet is typically a portion of something that is thin in comparison to its length and breadth. Preferably, of from 1 up to 8 sheets, more preferably of from 2 up to 5 sheets of material are stacked and are placed in the fluid vessel insertion device to prevent the proximal end of the puncture member from moving when in dry phase. For a given tensile strength of a certain material, the amount of sheets depend upon the size of the needle: the smaller the needle, the less resistance of the tissue to be punctured is experienced and the less pressure is put on the sheets of material in the dry phase. For the speed of retraction of the needle the system of layers of the same material is preferred over one layer which has the same thickness as all layers together. From experiments it was found that the stacking of layers gave a much higher retraction speed than the single thick layer.

The puncture member is shaped to allow fluid communication with the sheet of material. Typically, the puncture member is at least in part hollow, and hence comprises at least in part a lumen that allows for the passage of fluid therein. The puncture member may be elongate and/or tubular in form. The puncture member may be tapered towards a sharp end and may comprise a needle or trocar.

Advantageously, the retractor is an elastic object that stores mechanical energy, more preferably a spring. There are many spring designs, preferably a coil spring is being used. Springs can be made from a variety of elastic materials, the most common being spring steel. Some non-ferrous metals are also used including phosphor bronze and titanium for parts requiring corrosion resistance. In another preferred embodiment, the retractor might be in the form of a magnetic material. More preferably, the magnetic material might be incorporated in the piston head connected to the puncture member. In another preferred embodiment, a hydraulic configuration is being used as retractor.

Advantageously, the puncture member may be elongate in form and may have a longitudinal axis. Preferably, the puncture member comprises an opening at, or close by, or at a given distance to the side of the distal end, and an interior lumen in contact with the proximal end. The retraction mechanism may actuate the puncture member between the first and second conditions in a substantially axial direction. The distal end herein refers to the tip or end that is directed towards the vessel to be punctured, whereas the proximal end of the puncture member is inside the chamber, such that preferably the lumen is in fluid communication with the wettable sheet.

A cannulation sheet is the most common intravenous access method utilized in both hospitals and pre-hospital services. A peripheral cannulation sheet consists of a short catheter (a few centimetres long) inserted through the skin into a peripheral fluid vessel. This is usually in the form of a cannulation sheet over a puncture member, more preferably a flexible plastic cannula sheet comes mounted over a metal needle. Once the needle has retracted, the retraction system can be removed and discarded, leaving the cannulation sheet in place. Blood samples may be drawn directly after the initial cannulation sheet insertion. Any accessible fluid vessel, preferably a vein, more preferably a blood vein, can be used although arm and hand veins are used most commonly, with leg and foot veins used to a much lesser extent. Furthermore, arterial blood vessels, most commonly the radial artery, or surgically created arteriovenous conduits for hemodialysis access, can be used for cannulation.

The caliber of needles and catheters can be given in Birmingham gauge or French gauge. A Birmingham gauge of 14 is a very large cannula (used in resuscitation settings) and 24-26 is the smallest. The most common sizes are 16-gauge (midsize line used for blood donation and transfusion), 18- and 20-gauge (all-purpose line for infusions and blood draws), and 22-gauge (all-purpose pediatric line). 12- and 14-gauge peripheral lines are capable of delivering large volumes of fluid very fast, accounting for their popularity in emergency medicine.

The part of the catheter that remains outside the skin is called the connecting hub; it can be connected to a syringe or an intravenous infusion line, or capped with a heplock or saline lock, a needleless connection filled with a small amount of heparin or saline solution to prevent clotting, between uses of the catheter.

The dimensions of the chamber may vary depending on the fluid vessel that needs to be punctured and the treatments that are required. The chamber comprises the end of the puncture member, the sheet of material at the end of the puncture member and the retractor. The sheet of material is preferably fixed to the chamber, more preferably fixed using a glue, even more preferably using a clinically approved glue. The sheet of material is preferably fixed to the chamber via a supporting ring, more preferably via a supporting RVS ring. Advantageously, there might be a groove in the chamber where the sheet (and possible supporting RVS ring) can be placed against. Alternatively, the sheet of material is placed in a casing to hold it in a certain position. The casing might be made from any kind of material, it is preferably made from steel or from a polymer. It is also possible that a large part of the chamber is designed as one piece made of a polymer and made via a technique like injection moulding, that might include the sheet of material.

In a further preferred embodiment, the chamber may have a cavity at the end to allow the pressure within the system to be equalized to the surrounding pressure to facilitate the movement of fluid through the needle—the movement of fluid may not go as fast if the chamber is sealed off. Thus advantageously, it may have an opening in the wall to equalize pressure. This opening might be a dynamic opening in that it may be closed off by a mechanism to allow the opening to close when the system has retracted in order to prevent blood going through the opening when the vessel is punctured, and the needle retracted. In an alternative embodiment there might be a back-stop piece present that allows the ring and sheet to be pushed against the spring to prime the spring.

Embodiments of the invention are further described hereinafter with reference to the accompanying drawings, wherein like letters and numerals refer to like parts, wherein the figures are approximately to scale, and wherein:

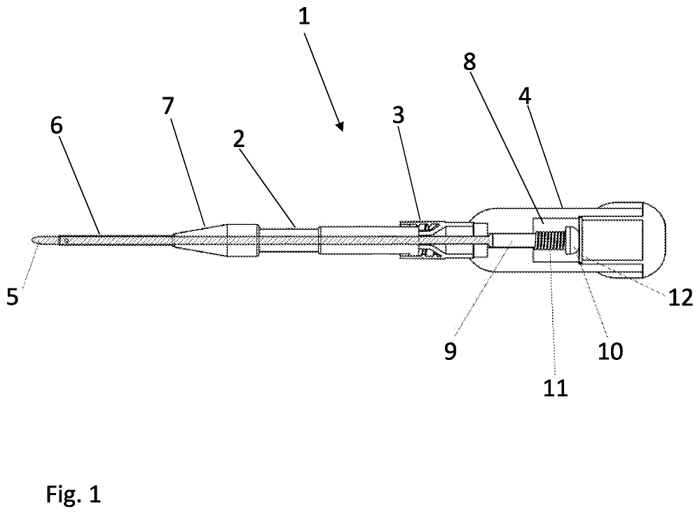

FIG. 1 illustrates an example of a fluid vessel insertion device in which the end of the puncture member protrudes beyond the end of the cannulation sheet;

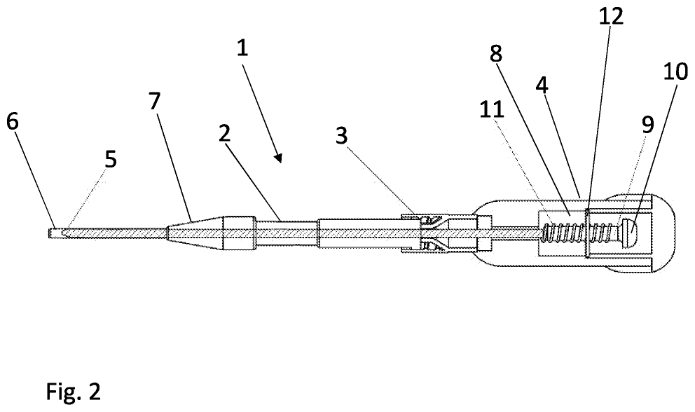

FIG. 2 illustrates an example of a fluid vessel insertion device in which the end of the puncture member is within the cannulation sheet;

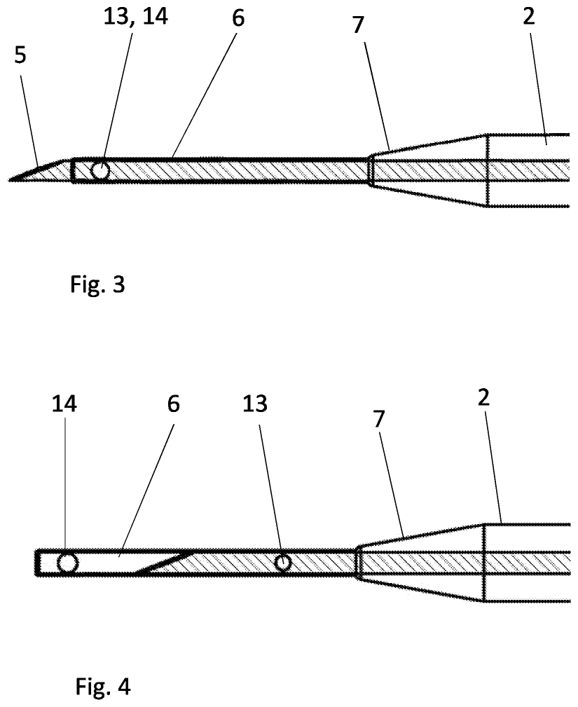

FIG. 3 illustrates an example of the punctuation device with a needle tip beyond the cannulation sheet with an opening in the punctuation device and in the cannulation sheet;

FIG. 4 illustrates an example of the punctuation device with a needle tip within the cannulation sheet with an opening in the punctuation device and in the cannulation sheet.

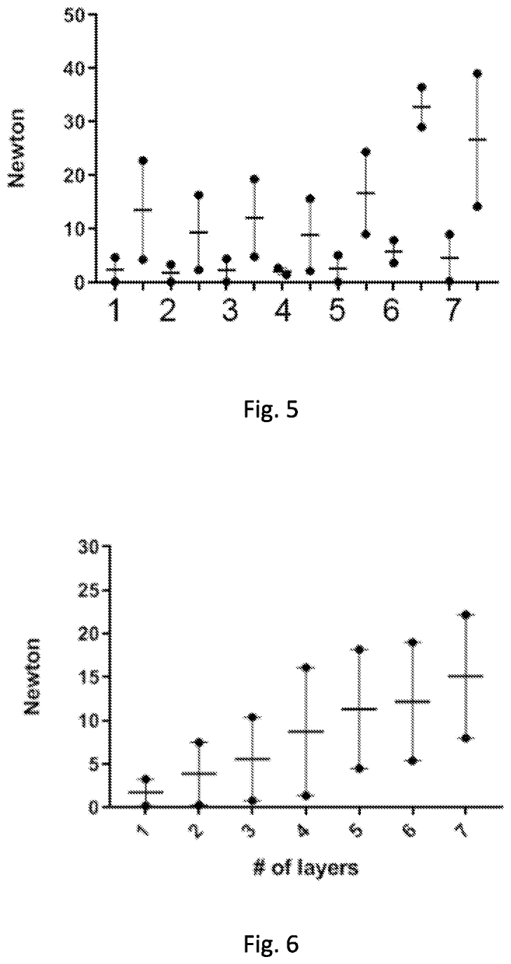

FIG. 5 shows the results of the comparison of the membrane tensile strength in dry and wet state of different sheets of cellulose membrane materials.

FIG. 6 shows the results of the comparison of the membrane layer thickness in dry and wet state, using an increasing number of sheet layers on top of each other.

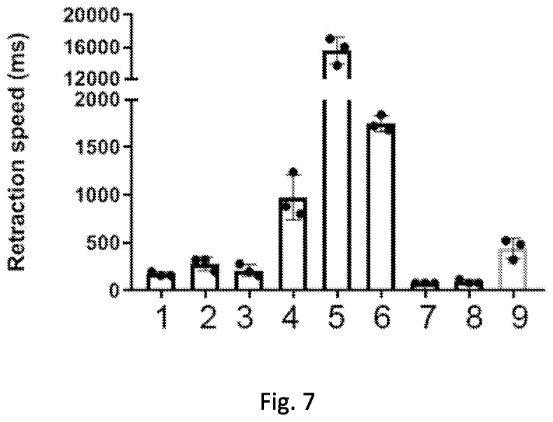

FIG. 7 shows a comparison of the retraction speed of various sheets, numbered 1 to 9, of different cellulose membrane materials, following wetting.

The invention described below is about an automatic mechanism that causes withdrawal of the puncture device out of the fluid vessel once a cannulation sheet is correctly positioned for communication. In medical applications, for insertion of a cannulation sheet into for example a blood vessel, the device is triggered when the needle moved into the blood vessel and contact is being made with blood. This removes the need for the person handling the device to manually retract the needle upon determination of the correct location of the needle in the blood vessel or vein. The automation of this part of the handling prevents a major cause of cannula insertion failure due to poor detection of the second wall of the blood vessel being punctured as well.

This invention may trigger automatic withdrawal of the puncture member, e.g. the needle, as soon as the cannulation sheet is inserted into the vessel based on the transport of the fluid through the needle wetting the wettable material outside of the vessel.

Whilst the invention finds particular use in the insertion of a cannulation sheet into one of the fluid vessels in the human body, it is not limited to vascular access applications and may be used in other instances where it is necessary to undertake such an operation with minimal loss of fluid and minimum discomfort of the patient where manual control of the operation is prone to potential failure.

It will be appreciated by the skilled person that the term “vessel” used herein may constitute a conduit, a cavity or a reservoir and should be construed accordingly.

Turning now to FIG. 1, this illustrates a first embodiment of an assembly (1) according to the invention, wherein the assembly (1) generally comprises a sleeve arrangement (2) attached via an attaching device (3) to housing (4). The sleeve arrangement (2) generally comprises a puncture member (5) and a cannulation sheet (6) attached to it, placed against a tapered wall (7). The sleeve arrangement (2) is connected via attaching device (3) to housing (4). The attaching device (3) is preferably a Luer taper. The Luer taper is a standardized system of small-scale fluid fittings used for making leak-free connections between a male-taper fitting and its mating female part on medical and laboratory instruments, including hypodermic syringe tips and needles or stopcocks and needles. Currently ISO 80369 governs the Luer standards and testing methods. Key features of Luer taper connectors are defined in the ISO 594 standards. There are two varieties of Luer taper connections: locking and slipping. Luer lock fittings are securely joined by means of a tabbed hub on the female fitting which screws into threads in a sleeve on the male fitting. Luer lock style connectors are divided into two types: one piece luer lock and two piece luer lock or rotating collar luer lock. One piece Luer lock comes as a single mold, and locking is achieved by rotating the entire luer connector or system. In two piece luer lock, a free rotating collar with threads is assembled to the luer and the locking is achieved by rotating the collar. Slip tip (Luer-slip) fittings simply conform to Luer taper dimensions and are pressed together and held by friction (they have no threads). Luer components are manufactured either from metal or plastic and are available from many companies worldwide. The attaching device (3) is more preferably a Luer taper of the locking type. The attaching device (3) is attached to housing (4) which comprises chamber (8). In chamber (8) the puncture member (5) with at the end a piston (9) with piston head (10) is present, as is a retractor (11) and a sheet of material (12). In the situation as presented in FIG. 1, the sheet of material (12) is in place and the retractor is in the initial position. The sharp end of the puncture member (5) is outside of the cannulation sheet (6) and can be used to puncture a fluid vessel. The retractor (11) is in a compressed position.

The sheet of material can be in any form, as long as the resistance of the material is decreased in such a way that the retractor can be moved in the longitudinal direction away from the direction of movement of the puncture member.

Turning to FIG. 2, it represents the situation were the sheet of material has been in contact with a fluid and is no longer withholding piston head (10), and the retractor (11) is in expanded form.

FIG. 3 illustrates a preferred embodiment of the current invention, wherein an opening (13) is present in the side of the puncture member (5) and an opening (14) is present in the cannulation sheet (6). The openings are placed on top of each other, in the start situation.

FIG. 4 illustrates the preferred embodiment of the current invention with the retractor (not visible) in extended form, the puncture member (5) has been retracted. The openings (13) and (14) are no longer on top of each other. In an alternative preferred embodiment, an opening (13) is present in the side of the puncture member only, placed more to the tip of the puncture member, such that in the starting position the opening is not covered by the cannulation sheet.

In an alternative preferred embodiment, an opening (13) is present in the side of the puncture member, and the tip at the distal end of the puncture member is sealed off.

The function of the side opening is to create retarded retraction. The stream of fluid in the fluid vessel enters the piston head not immediately when the vessel is punctured, but only after the puncture member is at the position that fluid can reach the opening. The puncture member is in this situation delayed in its retraction, and the puncture member remains longer in the initial position. This might be needed in some special cases, where it takes longer to place the cannulation sheet in the fluid vessel. The position of the side opening (13) of the puncture member is relative to the lumen of the to be punctured vessel. The distance between the tip at the distal end and the opening is advantageously determined by the thickness of the to be punctured vessel. In general the opening is advantageously placed at such a distance that the tip of the distal end does not touch the second wall of the vessel but the opening is within the vessel.

The present invention furthermore relates to a process for safely placing a cannulation sheet in a vessel, whereby a device comprising a puncture member is used, the puncture member being arranged within the cannulation sheet, whereby the puncture member in the first situation protrudes beyond an end of the cannulation sheet, and whereby the puncture member automatically retracts to a safe position within the cannulation sheet upon contact with a fluid by wetting a sheet of material in a chamber, whereby the sheet of material is comprising a material whose tensile strength is reduced upon contact with the fluid, and breaks under the pressure of the retractor.

The present invention also relates to the use of the device for safely inserting a cannulation sheet into a fluid vessel.

The following, non-limiting examples are provided to illustrate the invention. The use of sheets of wettable material is illustrated. As all samples were confidential, only sample numbers have been given. All samples comprised cellulose membrane materials in various compositions.

### Example 1

To assess the effectiveness of cellulose paper membranes, the properties with respect to tensile strength were tested using a number of different cellulose membrane materials. The tensile strength is important in the dry state to withhold the needle when pushing it through a tissue, e.g. the skin, and in the wet state it is preferably as low as possible so that a needle easily breaks through the material. Membrane properties were assessed in its dry (top data point) versus its wetted (bottom data point) state. The results of the tests are summarized in FIG. 5. Both a single sheet of cellulose paper membrane (the left dot for a material in the figure) and 4 sheets (the right dot for a material in the figure) on top of each other were tested. A piston was driven through the membranes at 50 mm·min, using a custom made probe burst set-up. The ultimate tensile strength was recorded. All experiments and iterations were performed in triplicate. Wetting the sheets was performed using 10 microliter of a phosphate buffered saline (PBS), which osmolarity and ion concentrations of the solution match those of the human body. Glycerol was added to the solution to mimic the viscosity of blood. As can be concluded from FIG. 5, different sheets of cellulose membrane materials give different results. Furthermore, the use of 4 layers of sheets is preferred over the use of 1 sheet, as the difference of tensile strength in this set-up between a dry and a wetted material is bigger.

### Example 2

Using the same set up and conditions as described in example 1, the number of sheets was tested using sample number 2, a cellulose membrane material. The results are given in FIG. 6. As can be seen from FIG. 6, the tensile strength decreases upon wetting for all the number of sheets used, with the biggest difference of tensile strength between dry and wetted state with 4 layers of sheet material.

### Example 3

Besides tensile strength, also the retraction speed is a factor that might be optimized. The retraction speed of the needle was assessed with various compositions of membranes. A custom made needle retraction device was placed in the pressure sensor of the tensile tester. The needle was primed using a membrane, and driven through a shore A60 silicone tube (wt:3 mm, ID: 6 mm) at a constant speed of 330 mm·min. The start coordinates of the system were known and recorded. The silicone lumen was filled with PBS with glycerol at 100 mmHg. The needle was allowed to puncture the silicone, causing fluid to enter the needle and induce retraction of the needle. The tensile tester software recorded the increase in pressure as the needle entered the silicone, as well as the drop in resistance following retraction, allowing the retraction speed to be determined. The results are given in FIG. 7. It can be concluded that the various sheets of cellulose membrane give various results and depending on the retraction speed designed, a material can be chosen.

1. A fluid vessel insertion device comprising
a) a puncture member arranged for location within a cannulation sheet and comprising an interior lumen;
b) a retractor arranged to actuate the puncture member relative to the cannulation sheet in use between
i) a first situation in which a distal end of the puncture member protrudes beyond an end of the cannulation sheet, and
ii) a second situation in which the distal end of the puncture member is within the cannulation sheet;
c) a chamber in fluid communication with an exterior of the device only via the lumen and distal end of the puncture member; and
d) a sheet of material located in the chamber to prevent the proximal end of the puncture member from moving;
wherein the distal end of the puncture member is configured as a puncturing end having a distal opening for insertion into a fluid vessel when in use, and
wherein the retractor triggers retraction of the puncture member between the first and the second situation automatically in response to wetting of the sheet of material by a fluid entering the puncture member lumen, whereby the sheet of material is comprising a material whose tensile strength is reduced upon contact with the fluid, and preferably breaks under the pressure of the retractor.

2. The device according to claim 1, wherein the sheet of material is comprising a wettable membrane, more preferably comprises cellulose or starch based membranes.

3. The device according to claim 1, wherein the sheet of material has a wet strength of less than 10%, preferably less than 8%.

4. The device according to claim 1, wherein of from 1 up to 8 sheets of material are stacked, more preferably of from 2 up to 5 sheets of material are stacked and are located in the chamber.

5. The device according to claim 1, wherein an opening is present in the side of the puncture member at a distance from the distal end.

6. The device according to claim 1, wherein an opening is present in the side of the cannulation sheet.

7. The device according to claim 1, wherein the retractor is a spring.

8. The device according to claim 1, wherein the sheet of material is fixed to the chamber, preferably fixed with a glue, more preferably fixed with a clinically approved glue.

9. The device according to claim 1, wherein the fluid vessel is a vein, preferably a blood vein.

10. A process for safely placing a cannulation sheet in a fluid vessel, whereby a device comprising a puncture member is used, the puncture member being arranged within the cannulation sheet, whereby the puncture member in the first situation protrudes beyond an end of the cannulation sheet, and whereby the puncture member automatically retracts to a safe position within the cannulation sheet upon contact with a fluid by wetting a sheet of material in a chamber, whereby the sheet of material is comprising a material whose tensile strength is reduced upon contact with the fluid, and breaks under the pressure of the retractor.

11. A process according to claim 10, wherein the fluid vessel is a vein, preferably a blood vein.

12. Use of a device according to claim 1 for placing a cannulation sheet into a fluid vessel, preferably a vein, more preferably a blood vein.
